(19) United States
(12) Patent Application Publication
(10) Pub. No.: US 2015/0328357 A1
(43) Pub. Date: Nov. 19, 2015

(54) WOUND PROTECTING POLYMERS

(71) Applicant: Joseph KENNEDY, Akron, OH (US)

(72) Inventor: Joseph Kennedy, Akron, OH (US)

(73) Assignee: THE UNIVERSITY OF AKRON, Akron, OH (US)

(21) Appl. No.: 14/761,669

(22) PCT Filed: Jan. 27, 2014
(86) PCT No.: PCT/US14/13126

§ 371 Date: Jul. 17, 2015

Related U.S. Application Data
(60) Provisional application No. 61/756,523, filed on Jan. 25, 2013.

(51) Int. Cl. A61L 15/24; C08F 255/08

(52) U.S. Cl. CPC A61L 15/24; C08F 255/08; A61L 2300/606

(57) ABSTRACT

A wound protecting composition includes an end-functionalized arborescent polymer having at least two branching points to provide at least four ends to the arborescent polymer, the arborescent polymer comprising polyisobutylene; and an end group attached to a majority of the ends of the arboresent polymer, the end group comprising an alkyl cyanoacrylate. The composition may further include a blend of the end-functionalized arborescent polymer with an independent alkyl cyanoacrylate.

### CROSS-REFERENCE TO RELATED APPLICATIONS

This application claims the benefit of U.S. Provisional Patent Application No. 61/756,523, filed Jan. 25, 2013, the disclosure of which is hereby incorporated by reference.

### FIELD OF THE INVENTION

This invention relates to wound protecting compositions, and more particularly, to wound protecting polymers made from arborescent polymers end functionalized or terminated with alkyl cyanoacrylates. The wound protecting polymers are more flexible and have a higher viscosity that similar wound protecting polymers such as polymerized 2-octyl cyanoacrylate.

### BACKGROUND OF THE INVENTION

There is a great need in biomedical applications, including orthopedic practice, for sealants of wounds and surgical cuts. Such sealants contemplated could range from sealants used for wound healing and wound closure on the skin to sealants used to permanently seal scalpel cuts and puncture wounds made by large bore injection needles in the course of various procedures.

Currently, the “gold standard” of today's wound-, incision-, or laceration-protecting compositions or materials is 2-octyl cyanoacrylate, sold commercially under the tradename Dermabond® and commercially available from Ethicon, Inc., of Somerville, N.J., a Johnson & Johnson company. 2-octyl cyanoacrylate (which hereinafter may be abbreviated as “Oct-CA”) is a very low viscosity liquid monomer that, when applied on the skin over a wound, rapidly (within seconds or minutes) polymerizes and cures to form a protective coating over the wound, laceration, or cut. The polymerization of Oct-CA (like cyanoacrylates in general) is initiated by nucleophiles (e.g., —NH₂, —OH groups) or moisture on the surface of a user's skin. Thus, the cyanoacrylate groups become bonded to the skin and the poly-Oct-CA surface coating becomes strongly attached to the skin.

It will be appreciated that there are numerous homopolymers of alkyl (methyl-, ethyl-, butyl-, octyl, etc.) cyanoacrylates that are well known to the art. Their use as adhesives (e.g., glues) has also been thoroughly investigated. For example, ethyl-cyanoacrylate, commonly known by its generic name Super Glue, is a well known adhesive, wherein the monomer is well understood, like the other lower (i.e, having 4 carbons or less) alkyl cyanoacrylates, to readily polymerize upon exposure to traces of moisture on various surfaces. However, these lower alkyl cyanoacrylates (particularly, methyl- and ethyl-cyanoacrylates) are toxic and cannot be used for most medical applications.

However, research has shown that the toxicity of cyanoacrylates diminishes upon increasing the length of the alkyl group attached directed to the cyanoacrylate group. Hence, a few years ago, the octyl derivative, 2-octyl cyanoacrylate, was approved for external use on skin by the U.S. Food and Drug Administration.

Nevertheless, while approved for external use on skin, Dermabond® (Oct-CA) has several shortcomings. One such deficiency of the Dermabond® composition is that it produces opaque stiff films that slough off easily from a user's skin shortly after application, particularly when the skin is in motion. The protective coatings, upon polymerizing, will typically become very stiff and lose most of its mechanical (e.g, tensile, elongation, etc.) properties within a few days from application. Because of this, the Dermabond® composition cannot be used where the skin is in frequent motion. Accordingly, it is not suitable for use around the mouth, over the elbow, knee, knuckles, wrist, ankle, and toes, and oftentimes cannot be used on the fingers, feet, or hips, wherein the skin will be stretched or pulled frequently. Applications at these sites would require a more flexible wound coating.

Another disadvantage of the Dermabond® composition is its very low viscosity. This monomer is very “runny”, i.e., it travels uncontrollably rapidly over surfaces and enters into cuts or wounds where it polymerizes. When this happens, the healing of the wound is delayed because the stiffened polymer that forms inside the wound or cut keeps the body from being able to heal itself or keeps topical medicines from reaching the necessary places within the wound, even if applied prior to application of the Dermabond® composition.

Still further, while the toxicity of the Dermabond® composition is sufficiently low for use on surfaces, it still is inappropriate and should not be used for under-the-skin applications. Only by substantially increasing the length of the alkyl substituent beyond octyl, will a moiety containing cyanoacrylate be suitable for such uses.

Thus, the need exists for a wound protecting composition that can overcome one or more of the above mentioned shortcomings of the Dermabond® composition. That is, the need exists for a wound protecting composition that is sufficient flexible to not slough off a user's skin when placed on a wound or cut of the user's body part that is frequently in motion. The need also exists for a wound protecting that has a higher viscosity such that it will coat, but will not flow into an open wound. The need also exists for a wound protecting composition that can be used in internal as well as external medical applications.

### SUMMARY OF THE INVENTION

The present invention is generally directed to a wound protecting composition, and more particularly, to a end-functionalized arborescent polymer. The wound protecting composition of the present invention provides for use of a highly-branched, high molecular weight polyisobutylene-based polymer terminated with cyanoacrylates, such that, when the composition is applied over a user's wound to his or her skin, the composition forms a flexible, transparent protective coating. Further, these cyanoacrylate-terminated polyisobutylene-based, highly-branched or arborescent polymer compositions are highly viscous liquids that will not flow into narrow cuts, lacerations or incisions on the user's skin. The viscosity is the wound protecting compositions of the present invention can be precisely controlled by controlling the molecular weight of the polyisobutylene moiety. Due to the rubberyness and flexibility of the polyisobutylene, the wound protecting composition that is formed over the wound should allow application where the user's skin is in frequent or constant motion, such as the elbow, knee, knuckle, finger, thumb, wrist, ankle, toe, foot, hip, etc. Moreover, because the wound protecting compositions use a hemo- and biocompatible-polyisobutylene (PIB) moiety, the toxicity of the composition, which still contains cyanoacrylates, will be further reduced such that the polyisobutylene-based polymer terminated with alkyl cyanoacrylates may be acceptable for under the skin applications.

These and other advantages of the present invention over existing prior art relating to wound protecting compositions, which shall become apparent from the description and drawings which follows, are accomplished by the invention as hereinafter described and claimed.

One or more aspects of the present invention may be achieved by a wound protecting composition comprising an end-functionalized arborescent polymer having at least two branching points to provide at least four ends to the arborescent polymer, the arborescent polymer comprising polyisobutylene; and an end group attached to a majority of the ends of the arborescent polymer, the end group comprising an alkyl cyanoacrylate. In one embodiment, the wound protecting composition (arb-PIB-t-CA) may include the end-functionalized arborescent polymer (denoted hereinafter sometimes as “arb-PIB” and as discussed herein below) as having a molecular weight of at least 2000 g/mol. In another embodiment, the wound protecting composition above may include the end-functionalized arborescent polymer as having a molecular weight of from about 2000 g/mol to about 50,000 g/mol. It will be appreciated that such a high molecular weight will increase and control the viscosity of the composition so as to make the composition sufficiently viscous such that it will not run or otherwise flow into any narrow wounds prior to polymerizing as would other compositions such as 2-octyl cyanoacrylate.

In another embodiment, the wound protecting composition above provides an arborescent polymer having a sufficient number of ends to provide for at least 5 end groups that comprise alkyl cyanoacrylates. Thus, it should be understood that a sufficient number of ends will be at least 6 in the case with the arborescent polymer provides for at least 5 end groups that contain alkyl cyanoacrylate (hereinafter sometimes referred to as “alkyl CA”). It would also mean that the end-functionalized arborescent polymer would need at least three branching points, but may have more than that, to accommodate the at least 5 end groups of alkyl CA. In another embodiment, the wound protecting composition above may provide an arborescent polymer having a sufficient number of ends to provide for at least 5 to about 30 end groups that comprise alkyl cyanoacrylates. In order to obtain 30 alkyl CA end groups, it will be appreciated that at least 30 ends on the polymer block are necessary. This would also mean that at last 15 branches of the arborescent polymer block would be necessary as well. It will be appreciated that in other embodiments, the arborescent polymer would have a sufficient number of ends to provide for at least 8, at least 10, at least 12, at least 15, at least 20 and at least 24 end groups.

In another embodiment, the wound protecting composition above provides for the end group as comprising an alkyl cyanoacrylate having at least 8 carbon atoms. In another embodiment, the wound protecting composition is provided wherein the end group is octyl cyanoacrylate.

In other embodiments, the wound protecting composition above provides the end-functionalized arborescent polymer as having a higher viscosity than 2-octyl cyanoacrylate. It will be appreciated that this is because the arborescent polymer has a much higher molecular weight than 2-octyl cyanoacrylate.

In at least one embodiment, the wound protecting composition above is known to adhere to skin within 5 minutes. In other embodiments, the wound protecting composition above will adhere to skin within 1 minute. In still other embodiments, the wound protecting composition above provides for an elastomeric end-functionalized arborescent polymer having a glass transition temperature of less than −40° C. In other embodiments, the glass transition temperature may be about −70° C.

Another aspect of the present invention provides for a wound protecting composition as above, that further includes independent alkyl cyanoacrylate that is separate from the alkyl cyanoacrylate present within the end-functionalized arborescent polymer. In at least one embodiment, the independent alkyl cyanoacrylate is blended with the end-functionalized arborescent polymer terminated with alkyl cyanoacrylate. In other embodiments, the wound protecting composition above provides for at least 2 carbon units on each alkyl unit of the independent alkyl cyanoacrylate. In other embodiments, the wound protecting composition above provides for at least 8 carbon units on each alkyl unit of the independent alkyl cyanoacrylate. In still other embodiments, the wound protecting composition above provides for the independent alkyl cyanoacrylate as being ethyl cyanoacrylate. In other embodiments, the wound protecting composition above provides for the independent alkyl cyanoacrylate as being octyl cyanoacrylate. Where ethyl cyanoacrylate is the independent alkyl cyanoacrylate, the ratio of the ethyl cyanoacrylate to the end-functionalized arborescent polymer is from about 5:1 to about 100:1. Where octyl cyanoacrylate is the independent alkyl cyanoacrylate, the ratio of the octyl cyanoacrylate to the end-functionalized arborescent polymer is from about 5:1 to about 250:1.

Another aspect of the present invention is achieved by use of the wound protecting composition as described above to protect a laceration or a cut to a user's skin by applying the wound protecting composition to the user's skin over the laceration or cut. It is to be noted that the wound protecting composition is more flexible than other wound protection compositions containing only 2-octyl cyanoacrylate. In one embodiment, the use above provides for a much more flexible wound protecting composition which can be more readily applied to a user's body part that is frequently in motion, such a body part being selected from an elbow, a knee, a knuckle, a finger, a thumb, a wrist, an ankle, a toe, a foot, a hip etc. As such, the wound protection composition will not become stiff and slough off within a day of application.

### BRIEF DESCRIPTION OF THE DRAWINGS

FIG. 1 is a representative schematic drawing of the arb-PIB-t-CA polymer of the present invention;

FIG. 2A is a prior art representative schematic drawing of a wound protecting composition on the skin made by polyOct-CA; and

FIG. 2B is a representative schematic drawing of a wound protecting composition made by arb-PIB-t-CA.

### DETAILED DESCRIPTION OF ILLUSTRATIVE EMBODIMENTS

As noted hereinabove, the present invention seeks to provide a novel wound protecting composition formed from an end-functionalized arborescent polymer. The end-functionalized arborescent polymer has at least two branching points to provide at least four ends to the polymer. In one embodiment, the arborescent polymer may have at least three branching points to provide at least 6 ends to the polymer. In other embodiments, the arborescent polymer may have at least 4, at least 5, at least 6, at least 10, or at least 15 branching points to provide a coordinated number of ends of the polymer. That is, a polymer with 4 branching points may have as few as 4 ends or as many as 8 ends. Similarly, a polymer having 5 branching points will have as few as 5 ends or a many as ten ends. Thus, it should be evident how many ends will be provided with any suggested number of branching points.

The arborescent polymer contains a polyisobutylene (“PIB”) moiety, and in one embodiment, may have a molecular weight of at least about 2000 g/mol. In another embodiment, the PIB moiety may have a molecular weight of at least 2500 g/mol. In other embodiments, the PIB may have a molecular weight of at least 4000 g/mol, at least 5000 g/mol, at least 6000 g/mol, at least 7500 g/mol, at least 10,000 g/mol, at least 20,000 g/mol, at least 30,000 g/mol, at least 40,000 g/mol or at least 50,000 g/mol. In yet another embodiment, the molecular weight of the PIB moiety may range from about 2000 g/mol to about 50,000 g/mol. It will be appreciated that the PIB moiety will thus constitute a substantial and predominant part of the arborescent polymer of the present invention.

In the present invention, an end group comprising an alkyl cyanoacrylate is attached to a majority of the ends of the arborescent polymer. By the term “majority,” it is meant that at least 50% of the ends of the arborescent polymer having alkyl CA attached to them. In another embodiment, at least 60% of the ends of the arborescent polymer have alkyl CA attached to them. In still other embodiments, the percent of alkyl CAs attached to the ends of the polymer may be at least about 70%, at least about 80%, at least about 90% or at least about 96%.

The wound protecting compositions include at least an end-functionalized arborescent polymer. This differs from other prior CA-terminated PIBs inasmuch as those prior PIBs were either linear or three-arm star PIBs with CA end groups. The present arbroescent polymers provides many more ends (i.e., more than three ends) for attachment of alkyl CA end groups, which is believed to provide a more desirable product, since more attachment points mean quicker bonding to the skin.

Generally it is well known how to terminate PIBs with CA groups, and particularly, alkyl CA groups. Preparation techniques have been disclosed in the earlier linear and three arm star patent application(s), such as disclosed in WIPO Publication No. WO 2012/109179, the disclosure of which is hereby incorporated by reference. The present invention provides for an arborescent polymer having a polymer block with at least two branching points and at least 4 ends. Thus, more ends are provided than by the three arm star polymer (which provides only 3 ends) previously disclosed in the WIPO application above.

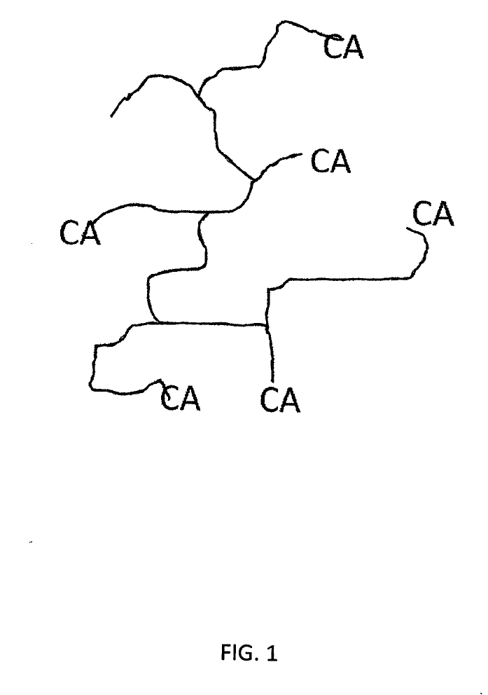

As noted above, the structure of the wound protecting polymer of the present invention may be abbreviated arb-PIB-t-CA, where arb indicates the arborescent structure of the polyisobutylene (PIB) moiety, and the t stands for telechelic, with the chain end being CA, which is an allyl cyanoacrylate. FIG. 1 shows the structure of the arb-PIB-t-CA, where the wavy lines indicate PIB chains and CA stands for allyl cyanoacrylate end groups (one of the end groups is occupied by the initiating residue, not shown).

The synthesis of arb-PIB-t-CA is accomplished in two steps. The first step is the synthesis of the precursor arborescent polymer, an arb-PIB whose end groups are fitted with tert Cl end functions, abbreviated arb-PIB-t-Cl. The preparation of an arb-PIB is generally set forth in U.S. Pat. No. 8,258,230, the disclosure of which is hereby incorporated by reference. The product is characterized by NMR spectroscopy and GPC. It is desirable to prepare an arb-PIB-t-Cl with a Mn in the 2,000-50,000 g/mol range because such a product will exhibit high viscosity but still can be squeeze-delivered or syringed. In the second step, the terminal —Cl groups of the arb-PIB-t-Cl are converted to CA end groups. The —Cl groups are first converted to —OH groups and the latter to —CA groups by quantitative dehydrochlorination and hydroboration/oxidation. Again, such techniques are known in the art as disclosed in various journal articles, including J. P. Kennedy, S. Midha, and A. Gadkari, “Macromers by Carbocationic Polymerization X. Synthesis, Characterization, and Polymerizability of Cyanoacrylate-Capped Polyisobutlyenes” J. Macromol. Sci.-Chem., A28, 2009-224 (1991); Younmoon Kwon and J. P. Kennedy, “Polymerizability, Copolymerizability and Properties of Cyanoacrylate-Telechelic Polyisobutylenes. I. Three-Arm Star Cyanoacrylate-Telechelic Polyisobutylene” Polymers for Advanced Technologies, 18, 800-807 (2007); and B. Ivan, J. P. Kennedy, and V. S. C. Chang, J. Polym. Sci., Polym. Chem. Ed., 18, 3177 (1980), the disclosures of which are incorporated herein by reference.

The molecular weight of arb-PIB-t-CA can be controlled by the polymerization conditions. In one embodiment, the molecular weight will be sufficiently high to obtain a reasonably high viscosity that is sufficient to prevent the wound protecting composition from flowing into the cuts, lacerations, or wounds to the user's skin. Such a molecular weight will be at least 2000 g/mol, and in one embodiment within a range of 2000 g/mol to about 10,000 g/mol. In another embodiment, the molecular weight may be at least 2500 g/mol. In other embodiments, the molecular weight may be at least 4000 g/mol, at least 5000 g/mol, at least 6000 g/mol, at least 7500 g/mol, at least 10,000 g/mol, at least 20,000 g/mol, at least 30,000 g/mol, at least 40,000 g/mol or at least 50,000 g/mol.

Further the arb-PIB-t-CA should contain a sufficient number of ends and CA end groups for rapid adhesion to the skin. Generally, it is believed that from 5 to 30 CA groups per arb-PIB-t-CA molecule will provide sufficient adhesion. It will be appreciated that the rate of adhesion to the skin is controlled by the number of CA groups in the arb-PIB-t-CA molecule. That is, the larger the number of CA groups, the faster the rate of adhesion (i.e., the shorter the set time). It is desirable to obtain good adhesion within 5 minutes and more preferably, within 1 minute. In other embodiments, it may be suitable to expect adhesion within 30 seconds, or even 20 seconds.

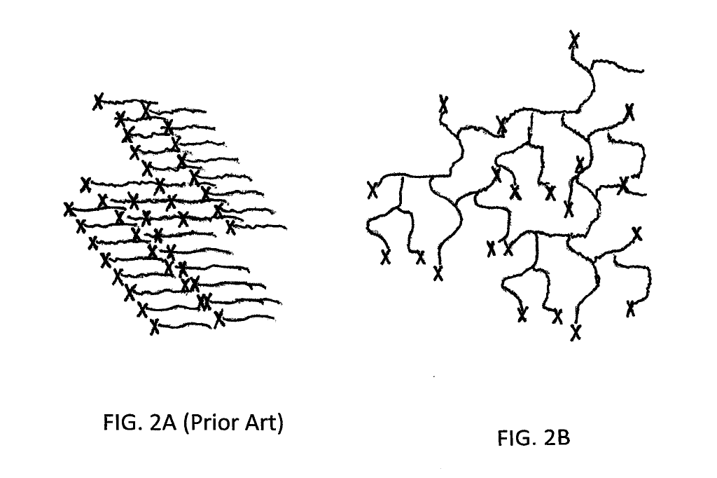

It will be appreciated that the structures of the wound protecting compositions produced by Oct-CA and arb-PIB-t-CA are fundamentally different. FIGS. 2A and 2B provides help in visualizing the molecular structure of the coating compositions on the skin made by poly Oct-CA and arb-PIB-t-CA. The lines indicate octyl groups or PIB chains, and the Xs indicate the sites where the CA groups adhere to the skin. FIG. 2A shows several arrays of poly Oct-CA molecules, while FIG. 2B shows three (intertwined) arb-PIB-t-CA molecules adhering to the skin as points X. The wound protecting composition produced by the arb-PIB-t-CA is elastomeric due to its rubbery PIB moiety and has with a glass transition temperature of at least −40° C., and more preferably, approximately −70° C. In contrast, the poly Oct-CA composition is stiff due to the relatively short octyl substituents and has a glass transition temperature of about 40° C.

It will be appreciated that arb-PIB-t-CA can be blended with a common low molecular weight alkyl cyanoacrylate, such as ethyl cyanocrylate (i.e., Super Glue), or octyl-cyanoacrylate. This low molecular weight alkyl cyanoacrylate would be separate and independent of the allyl cyanoacrylates attached to the ends of the branches of the polymer blocks of the arborescent polymer.

This composition noted above can be blended either mechanically or copolymerized. The use of such blends controls the set time (i.e., time for adhesion to skin) and viscosity of the mixture. Arb-PIB-t-CA and Oct-CA are miscible in bulk. Mixtures of arb-PIB-t-CA and ethyl-CA would need a compatibilizing solvent, such as THF, because these two are immiscible. The overall number of CA groups in the blend would be controlled by the concentration of the Oct-CA or ethyl-CA used, and the overall rate of adhesion time could be controlled. Thus, it is preferred that the ratio of ethyl-CA to arb-PIB-t-CA be from about 5:1 to about 100:1, and in another embodiment, from 10:1 to about 40:1, while the preferred ratio of octyl-CA to arb-PIB-t-CA be from about 5:1 to about 250:1, and in another embodiment, from about 10:1 to about 50:1.

It will be appreciated that the wound protecting composition can be used to protect a laceration or a cut to a user's skin comprising applying the wound protecting composition to the user's skin over the laceration or cut. Again, because it includes the PIB moiety, the wound protecting composition will be more flexible than other wound protection compositions containing only polymerized 2-octyl cyanoacrylate.

In one embodiment, the application of the wound protecting composition of the present invention can be accomplished by the use of prefilled plastic squeeze delivery vials or syringes. The content(s) in the squeeze delivery vial or syringe can be sterilize by heating the systems to about 125° C. for about one half hour. The sterile content(s) can then be delivered over the wound by squeezing the plastic container or pushing the syringe.

In another embodiment, the wound protecting composition of the present invention can be used in combination with a strip tape, such as a tape commercially available under the Tradename SeriStrip® from 3M Company of Minneapolis, Minn. The strip tape aids the composition in closing the cut, incision or wound. It is suggested to first close the cut with the strip tape and then apply the wound protecting composition subsequently over the strip tape. In one embodiment, biodegradable tape may be used.

In light of the foregoing, it should be appreciated that the present invention significantly advances the art by providing a wound protecting polymer that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A wound protecting composition comprising:
an end-functionalized arborescent polymer having at least two branching points to provide at least four ends to the arborescent polymer, the arborescent polymer comprising polyisobutylene; and
an end group attached to a majority of the ends of the arborescent polymer, the end group comprising an alkyl cyanoacrylate.

2. The wound protecting composition according to claim 1, wherein the end-functionalized arborescent polymer has a molecular weight of at least 2000 g/mol.

3. The wound protecting composition according to claim 2, wherein the end-functionalized arborescent polymer has a molecular weight of from about 2000 g/mol to about 10,000 g/mol.

4. The wound protecting composition according to claim 1, wherein the arborescent polymer has a sufficient number of ends to provide for at least 5 end groups that comprise alkyl cyanoacrylates.

5. The wound protecting composition according to claim 4, wherein the arborescent polymer has a sufficient number of ends to provide for at least 5 to about 30 end groups that comprise alkyl cyanoacrylates.

6. The wound protecting composition according to claim 1, wherein the end group comprises an alkyl cyanoacrylate having at least 8 carbon atoms.

7. The wound protecting composition according to claim 6, wherein the end group is octyl cyanoacrylate.

8. The wound protecting composition according to claim 6, wherein the end-functionalized arborescent polymer has a higher viscosity than 2-octyl cyanoacrylate.

9. The wound protecting composition according to claim 1, wherein the composition will adhere to skin within 5 minutes.

10. The wound protecting composition according to claim 1, wherein the composition will adhere to skin within 1 minute.

11. The wound protecting composition according to claim 1, wherein the end-functionalized arborescent polymer is elastomeric and has a glass transition temperature of about −70° C.

12. The wound protecting composition according to claim 1, further comprising independent alkyl cyanoacrylate, wherein the alkyl cyanoacrylate is blended with the end-functionalized arborescent polymer terminated with alkyl cyanoacrylate.

13. The wound protecting composition according to claim 12, wherein each alkyl unit of the independent alkyl cyanoacrylate has at least 2 carbon atoms.

14. The wound protecting composition according to claim 13, wherein the independent alkyl cyanoacrylate is ethyl cyanoacrylate.

15. The wound protecting composition according to claim 12, wherein each alkyl unit of the independent alkyl cyanoacrylate has at least 8 carbon atoms.

16. The wound protecting composition according to claim 15, wherein the independent alkyl cyanoacrylate is octyl cyanoacrylate.

17. Use of the wound protecting composition of claim 1 to protect a laceration or a cut to a user's skin comprising applying the wound protecting composition to the user's skin over the laceration or cut, wherein the wound protecting composition is more flexible than other wound protection compositions containing only polymerized 2-octyl cyanoacrylate.

18. The use according to claim 17, wherein the wound protection composition of claim 1 is applied to a user's body part that is frequently in motion, said body part being selected from an elbow, a knee, a knuckle, and a wrist, and wherein the wound protection composition will not become stiff and slough off within a day of application.
